(19) United States
(12) Patent Application Publication
(10) Pub. No.: US 2008/0195026 A1
(43) Pub. Date: Aug. 14, 2008

(54) Hemodialysis Tube Treated With Medicament on Surface Thereof For Connecting Artery to Vein

(76) Inventors: Dae Joong Kim, Seongnam-si (KR); Chul Soo Gim, Pyeongtaek-si (KR); Jai Young Ko, Pyeongtaek-si (KR); Hong Sang Park, Soeul (KR); Tae Gun Kwon, Seoul (KR); Byung Ha Lee, Gunpo-si (KR); Woo Kyoung Lee, Paju-si (KR); Sang Ho Jeong, Namwon-si (KR)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 11/628,503

(22) PCT Filed: Jun. 1, 2005
(86) PCT No.: PCT/KR05/01633

§ 371 Date: Dec. 15, 2006

(30) Foreign Application Priority Data
Jun. 10, 2004 (KR) 10-2004-0042660

(51) Int. Cl. A61M 37/00

(52) U.S. Cl. 604/6.16

(57) ABSTRACT

Disclosed herein is a hemodialysis tube treated with a medicament on the surface thereof, which provides stable communication between the artery and the vein of the patient under hemodialysis and can greatly reduce stenosis at arteriovenous connections. As a base material of the hemodialysis tube, expanded polytetrafluoroethylene is used while the medicament is paclitaxel or rapamycin.

### TECHNICAL FIELD

The present invention relates, in general, to a tube for use in connecting an artery to a vein upon hemodialysis and, more particularly, to a hemodialysis arteriovenous graft which provides stable communication between the artery and the vein of the patient under hemodialysis and can greatly reduce stenosis at arteriovenous connections.

### BACKGROUND ART

Generally, patients with serious renal failure are treated with hemodialysis. According to recent data, hemodialysis patients have been increasing in number.

For most hemodialysis patients, diabetic mellitus or hypertension is found to be a basic cause, entailing serious arteriosclerosis.

Successful hemodialysis requires the elimination of factors interrupting blood flowt at arteriovenous connections. Intensive studies have been conducted on such hemodialysis topics.

Artificial blood vessels have been developed to guide blood flow to compensate for the stenosis or significant dysfunction of real blood vessels. Depending on chemical compositions and physical properties, including porosity, elasticity, surface structure, etc., artificial blood vessels vary in patency.

Intensive attention has been paid to expanded polytetrafluoroethylene (e-PTFE) as a material for artificial blood vessels. A microporous thin film made by multi-axially drawing e-PTFE at high temperature and high pressure has such a low friction co-efficient as to show antithrombogenicity, e.g., not to allow proteins to adhere to the surface thereof.

Even though having advantages over autogenous arteriovenous fistula in performing hemodialysis in patients, artificial blood vessels that reduce the occurrence of stenosis at connections between arteriovenous vessels and the artificial graft need to be developed.

Blood vessels, whether artificial or in arteriovenous connections, are found to undergo stenosis because of the overgrowth of blood vessel endothelial cells. An arteriovenous graft, if narrowed, must be exchanged with a fresh one because successful hemodialysis may not be performed therethrough.

### DISCLOSURE OF INVENTION

### Technical Problem

Accordingly, the present invention has been made keeping in mind the above problems occurring in the prior art, and an object of the present invention is to provide a hemodialysis tube which can stably connect an artery to a vein in hemodialysis patients, thereby relieving blood vessel blockage.

### Technical Solution

In accordance with the present invention, the above object can be accomplished by the provision of a hemodialysis tube, which is an approximately cylindrical structure, treated with or comprising a medicament for inhibiting the overgrowth of blood vessel endothelial cells at its two opposite ends, whereby the hemodialysis tube can provide stable vascular access, serving as an arteriovenous fistula.

In the hemodialysis tube, the medicament is paclitaxel or rapamycin.

A feature of the present invention is that the medicament is either extruded together with the structural material or applied onto the surface of the structure.

The hemodialysis tube is characterized in that the structure comprises a microporous thin film made from expanded polytetrafluoroethylene. Preferably, the structure is made from Gore-tex.

In the hemodialysis tube, the structure is treated with a medicament on both the outer and the inner surface thereof at at least the junction to a blood vessel.

The structure has multilayer arrangement comprising a medicament layer and a microporous layer, or a first medicament layer, a first microporous layer, a second medicament layer and a second microporous layer in order on each surface thereof, with a medicament layer in direct contact with the structure, the microporous layer being suitable for releasing the medicament in a controlled manner.

In the hemodialysis tube, the structure is treated with the medicament on an inner surface thereof or on both an inner and an outer surface of thereof, to form a medicament layer ranging in thickness from 1 to 10 μm.

In the hemodialysis tube, the structure is treated with or comprises the medicament in an amount of 10 to 500 μg per cm2 of surface area over the entire surface.

The medicament is applied both on an inner surface and an outer surface of the structure.

### Advantageous Effects

When an autogenous blood vessel of a hemodialysis patient cannot be used as a bypass upon hemodialysis, the hemodialysis tube of the present invention can be an arteriovenous graft through which stable hemodialysis can be performed. In addition, the hemodialysis tube of the present invention can prevent the overgrowth of blood vessel endothelial cells due to the paclitaxel or rampamycin applied thereto, which results in the prevention of edema or stenosis at the arteriovenous connections and reduced in patient pain and production cost.

### BRIEF DESCRIPTION OF THE DRAWINGS

FIG. 1 is a schematic view showing an arteriovenous graft in a hemodialysis patient;

FIG. 2 is a cross sectional view of a hemodialysis tube in accordance with an embodiment of the present invention; and

FIG. 3 is a cross sectional view of a hemodialysis tube in accordance with another embodiment of the present invention.

### BEST MODE FOR CARRYING OUT THE INVENTION

Reference should now be made to the drawings, in which the same reference numerals are used throughout the different drawings to designate the same or similar components.

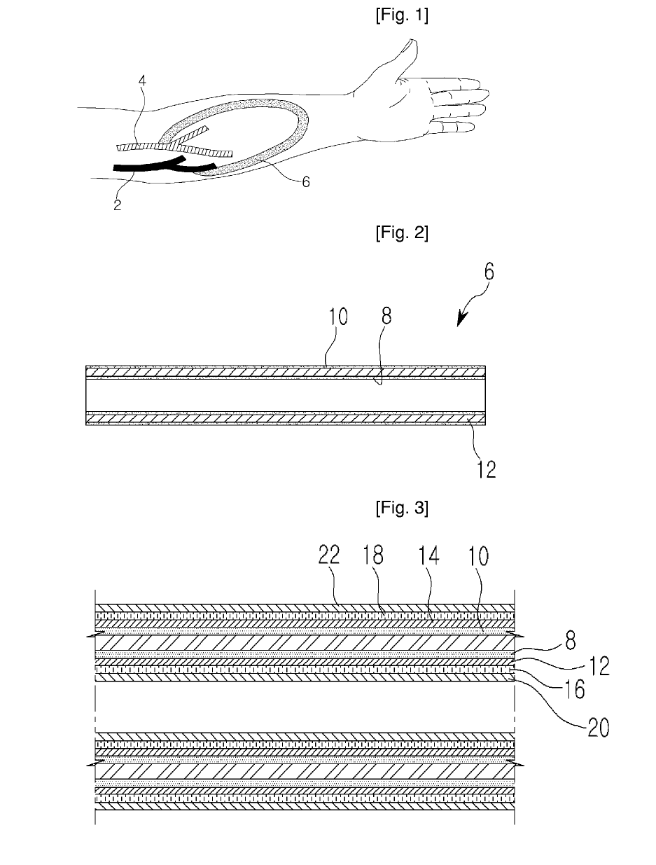

FIG. 1 schematically shows a tube provided for connecting an artery to a vein to dialyze the blood of a hemodialysis patient in accordance with the present invention, and the arteriovenous tube is shown in a cross sectional view in FIG. 2.

Typically, arteriovenous fistula creation, as seen in FIG. 1, is performed by subcutaneously incising a predetermined position of the body, cutting an artery 2 and a vein 4, joining a tube 2 to both the artery 2 and the vein 4 at the cut positions, and suturing the incised portions. In addition to capillary vessels, the arteriovenous graft can be used as a bypass through which blood can be dialyzed and as a capillary vessel that connects an artery to a vein.

After being provided for connecting the artery 2 and the vein 4, the tube 6 is connected through a needle to a blood dialyzer.

As a bypass, a patient's autogenous vessel may be used. In many cases, however, a patient cannot utilize autogenous vessels because the vessels are or become dysfunctional.

Advantageous as they are over autogenous vessels in some aspects, such arteriovenous grafts have significant problems to be solved. That is, at the junctions between the tube and the vessels, vessel endothelial cells overly grow to produce edema or to narrow the vessels, so that the arteriovenous grafts cannot function as a bypass.

In this stenosis condition, hemodialysis is impossible, thereby an operation for installing a shunt is required. The present invention provides an arteriovenous graft as a bypass which is not narrowed.

In accordance with an embodiment of the present invention, a hemodialysis tube 6 is provided which comprises a base layer 12 and two medicament layers 8 and 10 coated respectively on an inner and an outer surface of the base layer 12, as shown in FIG. 2, at least at opposite terminal portions which are connected to vessels. Of course, the medicament layers 8 and 10 may be formed over the entire surface of the base layer 12. Each of the medicament layers 8 and 10 includes paclitaxel or rapamycin as a functionally effective ingredient and the base layer 12 is a microporous e-PEFE thin film which can be obtained by multi-axially drawing PTFE at a high pressure and a high temperature.

More preferably, the base layer 12 can be prepared by extruding Gore-tex into a cylindrical form. The application of the medicament is achieved by extruding a mixture of a Gore-tex material and the medicament. Alternatively, the medicament may be applied to both sides of the cylindrical Gore-tex layer after the extrusion.

After being treated on both surfaces of the base layer, the medicament layer preferably ranges in thickness from 1 to 10 μm with a density of 10 to 500 μg per cm2 of each surface. Within the ranges of thickness and density, the medicament can provide medicinally useful effects without the interruption of blood currents.

In accordance with another embodiment, a microporous layer is provided on the medicament layer directly in contact with the surface of the base tube so as to control the release of the medicament. The microporous layer may be made from a material identical to or different from the base tube.

Accordingly, the present invention provides a multilayer hemodialysis tube in which a microporous layer and a medicament layer are formed in an alternating manner on each of the inner and the outer surface of a base tube, with a medicament layer in direct contact with the base tube, and a microporous layer present as an outermost layer. As shown in FIG. 3, a hemodialysis tube in accordance with an embodiment has a base tube, on each surface of which a first medicament layer 8, 10, a first microporous layer 12, 14, a second medicament layer 16, 18 and a second microporous layer 20, 22 are laminated in order. In another embodiment of the present invention, the second medicament layer 16, 19 and the second microporous layer 20, 22 are omitted.

With such a multilayer structure, the hemodialysis tube for arteriovenous connection allows the medicament layer to be in contact with the artery and vein vessels to which the hemodialysis tube is applied, thereby showing highly improved long term patency.

### INDUSTRIAL APPLICABILITY

As described hereinbefore, the hemodialysis tube of the present invention can stably connect an artery to a vein therethrough in hemodialysis patients, with an improvement in blood vessel blockage. Accordingly, the present invention is very useful for patients who are forced to undergo periodic hemodialysis.

1. A hemodialysis tube, which is an approximately cylindrical structure treated with or comprising a medicament for inhibiting the overgrowth of blood vessel endothelial cells, at its two opposite ends, whereby the hemodialysis tube can be used as a stable vascular arteriovenous fistula.
The hemidialysis tube as set forth in claim 1, wherein the medicament is paclitaxel or rapamycin.
The hemodialysis tube as set forth in claim 1, wherein the structure comprises a microporous thin film made from expanded polytetrafluoroethylene.
The hemodialysis tube as set forth in claim 1, wherein the structure comprises a Gore-tex tube.
The hemodialysis tube as set forth in claim 1, wherein the structure is treated with a medicament on both the outer and the inner surfaces thereof at least at junctions to blood vessels.
The hemodialysis tube as set forth in claim 1, wherein the structure has a multilayer arrangement comprising a medicament layer and a microporous layer, or a first medicament layer, a first microporous layer, a second medicament layer and a second microporous layer in order, on each surface thereof, with a medicament layer in direct contact with the structure, said microporous layer being suitable for releasing the medicament in a controlled manner.
The hemodialysis tube as set forth in claim 1, wherein the structure is treated with the medicament on an inner surface thereof or on both an inner and on an outer surface of thereof, to form a medicament layer ranging in thickness from 1 to 10 μm.
The hemodialysis tube as set forth in claim 1, wherein the structure is treated with or comprises the medicament in an amount of 10 to 500 μg per cm2 of surface area over an entire surface.
The hemodialysis tube as set forth in claim 1, wherein the medicament is applied on both an inner surface and an outer surface of the structure.
The hemodialysis tube as set forth in claim 1, wherein the medicament is extruded together with a structural material or applied onto a surface of the structure.
